(19) United States
(12) Patent Application Publication
(10) Pub. No.: US 2020/0093855 A1
(43) Pub. Date: Mar. 26, 2020

(54) ENHANCED IMMUNE RESPONSE UPON TREATMENT WITH NITRIC OXIDE

(71) Applicants: Christopher C. Miller, North Vancouver (CA); Gilly Regev, Vancouver (CA)

(72) Inventors: Christopher C. Miller, North Vancouver (CA); Gilly Regev, Vancouver (CA)

(21) Appl. No.: 16/430,354

(22) Filed: Jun. 3, 2019

Related U.S. Application Data
(63) Continuation of application No. 15/701,363, filed on Sep. 11, 2017, now abandoned.

(51) Int. Cl. A61K 33/00; A61K 31/365

(52) U.S. Cl. CPC A61K 33/00; A61K 31/365

(57) ABSTRACT

The present invention relates to compositions and methods useful for immune activation that is effective for eliciting a non-antigen-specific immune response in a subject. An immunomodulator composition can include a therapeutically effective amount of a liquid nitric oxide releasing solution (NORS) for eliciting an immune response in a subject to treat an adverse health condition in the subject.

### PRIORITY DATA

This application is a continuation of U.S. patent application Ser. No. 15/701,363, filed Sep. 11, 2017, which is incorporated herein by reference.

### BACKGROUND

Mammalian organisms and other species are susceptible to many types of viral, bacterial, fungal, and parasite infections. Non-limiting examples can include central nervous system infections, skin infections, ear infections, eye/eyelid infections, respiratory tract infections, gastrointestinal tract infections, bone/joint infections, heart infections, urinary tract infections, etc. Current prevention and treatment of such infections generally consists of vaccination against viruses and bacteria and antimicrobial therapy for sick subjects. However, many vaccines are primarily intended to prevent disease and do not necessarily protect against infection. Thus, in some cases, effectiveness can depend on the specific match between the vaccine and the virus or bacteria infecting the host. Additionally, conventional treatments for sick subjects include the administration of antibiotics to treat or control infections. Yet, in many cases, not only is there a bacterial infection, but also a viral infection. As such, in some cases, both vaccines and antibiotics can be ineffective at preventing and treating infectious diseases.

### BRIEF DESCRIPTION OF THE DRAWINGS

Invention features and advantages will be apparent from the detailed description which follows, and are further enhanced in conjunction with the accompanying drawings, which together illustrate, by way of example, various invention embodiments; and, wherein:

FIG. 1A is a graph showing incidence of BRDc in bovine after 7 and 14 days post arrival to feedlot in one example.

FIG. 1B is a graph showing incidence of BRDc in bovine after 7 and 14 days post arrival to feedlot in one example.

FIG. 2A illustrates changes in expression of key innate immune genes following BHV-1 infection. The gene expression levels in the non-infected controls were used as a baseline against which to measure percentage changes in gene expression. T1 was measured 30 hrs post BHV-1 infection (White), T2 was measured 46 hrs post BHV-1 infection (Gray); p<0.05=(*), p<0.01=(**), p<0.001=(***).

FIG. 2B illustrates changes in expression of key innate immune genes following culturing with LPS. The gene expression levels in the non-infected controls were used as a baseline against which to measure percentage changes in gene expression. T1 was measured 4 hrs post addition of LPS (White), T2 was measured 20 hrs post addition of LPS (Gray); p<0.05=(*), p<0.01=(**), p<0.001=(***).

FIG. 2C illustrates changes in expression of key innate immune genes following BHV-1 infection and subsequent culturing with LPS (BRDc model). The gene expression levels in the non-infected controls were used as a baseline against which to measure percentage changes in gene expression. T1 was measured 30 hrs post BHV-1 infection/4 hrs post addition of LPS (White), T2 was measured 46 hrs post BHV-1 infection/20 hrs post addition of LPS (Gray); p<0.05=(*), p<0.01=(**), p<0.001=(***).

FIG. 3 illustrates changes in protein levels of key innate immune genes following BHV-1 infection, culturing with LPS or a combination of both. The protein levels in the non-infected controls were used as a baseline against which to measure percentage changes in protein release. T1 was measured 30 hrs post BHV-1 infection/4 hrs post addition of LPS (White), T2 was measured 46 hrs post BHV-1 infection/20 hrs post addition of LPS (Gray); p<0.05=(*), p<0.01=(**), p<0.001=(***).

FIG. 4A illustrates changes in IL-1β expression levels in response to NORS treatment of PBMCs under various experimental conditions: ( [symbol] ) Non-infected Cell Control, ( [symbol] ) BHV-1 infected, ( [symbol] ) LPS cultured and both BHV-1 infected and LPS cultured ( [symbol] ). The gene expression/protein levels in the non-treated controls were used as a baseline against which to measure percentage changes in signal due to NORS. The gene expression and protein levels were measured at T1 28 hrs post NORS intervention and T2 44 hrs post NORS intervention; p<0.05=(*), p<0.01=(**), p<0.001=(***).

FIG. 4B illustrates changes in the IL-1β protein levels in response to NORS treatment of PBMCs under various experimental conditions: ( [symbol] ) Non-infected Cell Control, ( [symbol] ) BHV-1 infected, ( [symbol] ) LPS cultured and both BHV-1 infected and LPS cultured ( [symbol] ). The protein levels in the non-treated controls were used as a baseline against which to measure percentage changes in signal due to NORS. The protein levels were measured at T1 28 hrs post NORS intervention and T2 44 hrs post NORS intervention; p<0.05=(*), p<0.01=(**), p<0.001=(***).

FIG. 4C illustrates changes in TNF expression levels in response to NORS treatment of PBMCs under various experimental conditions: ( [symbol] ) Non-infected Cell Control, ( [symbol] ) BHV-1 infected, ( [symbol] ) LPS cultured and both BHV-1 infected and LPS cultured ( [symbol] ). The gene expression levels in the non-treated controls were used as a baseline against which to measure percentage changes in signal due to NORS. The gene expression levels were measured at T1 28 hrs post NORS intervention and T2 44 hrs post NORS intervention; p<0.05=(*), p<0.01=(**), p<0.001=(***).

FIG. 4D illustrates changes in TNF protein levels in response to NORS treatment of PBMCs under various experimental conditions: ( [symbol] ) Non-infected Cell Control, ( [symbol] ) BHV-1 infected, ( [symbol] ) LPS cultured and both BHV-1 infected and LPS cultured ( [symbol] ). The protein levels in the non-treated controls were used as a baseline against which to measure percentage changes in signal due to NORS. The protein levels were measured at T1 28 hrs post NORS intervention and T2 44 hrs post NORS intervention; p<0.05=(*), p<0.01=(**), p<0.001=(***).

FIG. 5 illustrates Changes in TLR4 expression levels in response to NORS treatment of PBMCs under various experimental conditions: ( [symbol] ) Non-infected Cell Control, ( [symbol] ) BHV-1 infected, ( [symbol] ) LPS cultured and both BHV-1 infected and LPS cultured ( [symbol] ). The gene expression levels in the non-treated controls were used as a baseline against which to measure percentage changes in TLR4 gene expression due to NORS. The gene expression levels were measured at T1 28 hrs post NORS intervention and T2 44 hrs post NORS intervention; p<0.05=(*), p<0.01=(**), p<0.001=(***).

FIG. 6A illustrates levels (pg/ml) of IFN-γ in nasal secretions after treatment with either nitric oxide releasing solution (NORS) (red line), antibiotic (Draxon) (black line), or saline (blue line).

FIG. 6B illustrates levels (pg/ml) of IFN-α in nasal secretions after treatment with either nitric oxide releasing solution (NORS) (red line), antibiotic (Draxon) (black line), or saline (blue line).

These figures are provided to illustrate various aspects of certain invention embodiments and are not intended to be limiting in scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a subject” includes a plurality of subjects.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, “subject” refers to a mammal that may benefit from the administration of NORS. In one aspect, the mammal may be a human.

As used herein, the terms “treat,” “treatment,” or “treating” when used in conjunction with the administration of NORS, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition. Further, these terms can encompass metaphylactic acts of administering NORS to bovine in anticipation of an expected outbreak of disease. Moreover, a “treatment outcome” refers to a result obtained at least in part, due to behavior or an act taken with regard to a subject. Treatment outcomes can be expected or unexpected. In one specific aspect, a treatment outcome can be a delay in occurrence or onset of a disease or conditions or the signs or symptoms thereof.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a solution that releases nitric oxide.

As used herein “NORS” refers to a nitric oxide (NO) releasing solution, composition or substance. In one aspect, NO released from NORS may be a gas.

As used herein a “therapeutic agent” refers to an agent that can have a beneficial or positive effect on a subject when administered to the subject in an appropriate or effective amount. In one aspect, NO can be a therapeutic agent.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen,” or a “metaphylactic dosing regimen” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as NO, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as NO, from a composition or formulation.

As used herein, the term “controlled release” refers to non-immediate release of an agent or substance, including a therapeutic agent, such as NO, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein, the term “elicit” can be used interchangeably with the terms activate, stimulate, generate or upregulate.

As used herein, the term “eliciting an immune response” in a subject refers to specifically controlling or influencing the activity of the immune response, and can include activating an immune response, upregulating an immune response, enhancing an immune response and/or altering an immune response (such as by eliciting a type of immune response which in turn changes the prevalent type of immune response in a subject from one which is harmful or ineffective to one which is beneficial or protective). As used herein, the term “cytokine” refers to an immune enhancing protein family. The cytokine family includes hematopoietic growth factor, interleukins, interferons, immunoglobulin superfamily molecules, tumor necrosis factor family molecules and chemokines (i.e. proteins that regulate the migration and activation of cells, particularly phagocytic cells). Exemplary cytokines include, without limitation, interleukin-2 (IL-2), interleukin-12 (IL12), interleukin-15 (IL-15), interleukin-18 (IL-18), interferon-a (IFNa), and interferon-y (IFNy).

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 ml to about 80 ml” should also be understood to provide support for the range of “50 ml to 80 ml.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Nitric oxide (NO) is a naturally occurring nano-molecule that plays a major role in a variety of physiological processes including modulation of wound healing, vasodilation, neurogenesis, angiogenesis and is both a modulator and effector of the host innate immune response. A variety of immune cells (such as dendritic cells, NK cells, macrophages, mast cells, eosinophils, neutrophils, and T cells, for example) produce, and respond to, NO. The NO released by these cell types has a multifunctional concentration dependent role in the immune response which includes but is not limited to antimicrobial; both tumoricidal and tumorigenic; pro- and anti-inflammatory and immunomodulatory activities. As an antimicrobial, NO can also act directly as a nitrosative agent and indirectly on foreign microbes through the formation of cytotoxic reactive intermediate species which cause damage to pathogens through various mechanisms including DNA alteration and enzyme function inhibition.

More specifically, NO can work by multiple mechanisms of action. For example, NO can work by at least the following mechanisms of action: 1) The antimicrobial action of the exogenous NO helps alter the viral and/or pathogenic microbiome in the nasal cavity and upper respiratory tract through direct cytotoxic interaction with, and killing of, invading pathogens. 2) With a strong proinflammatory response induced by the active infection, the high concentrations of exogenous NO act to inhibit inflammation in order to limit immunopathogenesis. This occurs in conjunction with an increase in TLR4 expression suggesting a dual action mechanism mediated by the exogenous NO in which harmful inflammation is decreased, limiting tissue damage while simultaneously enhancing the ability of the host to detect pathogens. 3) Additionally, the exogenous NO modulates the adaptive response through selective proliferation of anti-inflammatory promoting T cells in the periphery. This results in the expansion of a specific immune response in an environment in which inflammation is controlled, alleviating clinical symptoms and providing prolonged host protection.

Further, NO is a free-radical which is lipophilic with a small stokes radius making it an excellent signaling molecule enabling it to readily cross the plasma membrane into the cytosol, and is therefore believed to be suitable for treatment of a variety of indications. Non-limiting examples can include the common cold, sinusitis, tonsillitis, pharyngitis, epiglottitis, laryngotracheitis, bronchitis, bronchiolitis, pneumonia, the flu (e.g. swine flu, avian flu, etc.), respiratory syncytial virus (RSV), tuberculosis, pertussis, enterovirus, severe acute respiratory syndrome (SARS), middle east respiratory syndrome (MERS), chronic obstructive pulmonary disease (COPD), the like, or combinations thereof.

Accordingly, invention embodiments relate to formulations and methods for eliciting an immune response in a subject to help combat at least one adverse health condition. Such embodiments can include administration of a therapeutically effective amount of an immunomodulator composition to elicit an immune response. The immunomodulator composition can include a liquid nitric oxide releasing solution (NORS) as a vehicle for releasing an effective amount of gaseous nitric oxide (gNO) to a site or situs of administration and/or to a targeted treatment site or situs that is distal to the administration site. In addition, the immunomodulator elicits a non-antigen-specific immune response that is effective alone or enhances the operation of at least one biological agent such as a vaccine or antimicrobial therapeutic, when administered prior to such a biological agent, co-administered with such a biological agent, administered after a biological agent, or mixed with the biological agent.

The invention embodiments of the current technology provide new treatment options and strategies for protecting subjects from infectious diseases and treating populations having infectious disease. Additionally, the invention embodiments described herein can provide a more rapid, a longer, and better protection against a disease when the immunomodulator is used in combination with a biological agent.

### Composition

a. Immunomodulator

In one embodiment of the invention, the immunomodulator composition includes a therapeutically effective amount of a liquid NORS for eliciting an immune response in a subject to treat an adverse health condition. In one embodiment, the NORS can include the use of water or a saline-based solution or substance and at least one NO releasing compound, such as nitrite or a salt thereof. In one embodiment, the NORS is a saline-based solution or substance. In one embodiment, the NO releasing compound is a nitrite, a salt thereof, or any combinations thereof. Non-limiting examples of nitrites include nitrite salts such as sodium nitrite, potassium nitrite, barium nitrite, and calcium nitrite, mixed salts of nitrite such as nitrite orotate, and nitrite esters such as amyl nitrite. In one embodiment, the NO releasing compound is selected from the group consisting of sodium nitrite and potassium nitrite, or any combinations thereof. In another embodiment, the NO releasing compound is sodium nitrite. In one embodiment, the NORS can comprise a sodium nitrite in a saline solution. In another embodiment, the solution can comprise a potassium nitrite in a saline solution.

In one embodiment, the concentration of NO releasing compound, for example, nitrite (i.e. NO₂), in the NORS can be from 0.07% w/v to about 1.0% w/v. In one embodiment, the concentration of nitrites in the solution is no greater than about 0.5% w/v. In another embodiment, the concentration of nitrites in the solution is about 0.1% w/v. In a further embodiment, the concentration of nitrites in the solution is about 0.2% w/v. In an additional embodiment, the nitrite concentration is about 0.3% w/v. In another embodiment, the nitrite concentration is about 0.4% w/v. In yet another embodiment, the concentration of nitrite in the solution is about 0.28% w/v. In an additional embodiment, the nitrite concentration in the solution is about 0.32% w/v. In an additional embodiment, the nitrite concentration in the solution is about 0.38% w/v. In another embodiment, the nitrite concentration in the solution is about 0.41% w/v. In a further embodiment, the nitrite concentration in the solution is about 0.46% w/v. In another embodiment, the nitrite concentration in the solution is from about 0.07% w/v to about 0.5% w/v. In a further embodiment, the nitrite concentration in the solution can be from about 0.05% w/v to about 10% w/v. As used herein, the term “w/v” refers to the (weight of solute in grams/milliliters of volume of solution)×100%. In one embodiment, when sodium nitrite is used in the solution, the concentration of sodium nitrite can be from about 0.41% w/v to about 0.69% w/v. Other nitrite salts can be used as a source of NO₂ and the specific amount of each required to provide appropriate NO₂ concentrations and concentration ranges as herein described can be determined by one of ordinary skill in the art in view of the present disclosure.

In an additional embodiment, the amount of NO releasing agent, for example nitrite (i.e. NO₂), can be a concentration of from about 1 mM to about 1M. In another embodiment, the nitrite concentration can be from about 10 mM to about 500 mM. In yet a further embodiment, the nitrite concentration in the solution can be from about 100 mM to about 200 mM. In an additional embodiment, the nitrite concentration in the solution can be from about 40 mM to about 180 mM. In a further embodiment, the nitrite concentration in solution can be about 160 mM. In an additional embodiment, the nitrite concentration in solution can be from about 40 mM to about 120 mM. In another embodiment, the nitrite content can be from about 51 mM to about 100 mM. In another embodiment, the nitrite concentration can be about 60 mM. In yet another embodiment, the concentration can be 100 mM. In an additional embodiment the concentration of nitrite in the solution can be about 109 mM or less. In a further embodiment, when sodium nitrite is used in the solution, the concentration of sodium nitrite can be about 72 mM. Again, other nitrite salts can be used as a source of NO₂ and the specific amount of each required to provide appropriate NO₂ concentrations and concentration ranges as herein described can be determined by one of ordinary skill in the art in view of the present disclosure.

In one embodiment, the NORS can also contain at least one acidifying agent. As described elsewhere herein, the addition of at least one acidifying agent to the NORS solution contributes toward increased production (i.e. attenuates production) of NO from the NORS solution or substance. Any acidifying agent which contributes to NO production is contemplated by the present technology. In one embodiment, the acidifying agent can be an acid. In one aspect, the acid can be an organic acid. In another aspect, the acid can be an inorganic acid. Non-limiting examples of acids include ascorbic acid, salicylic acid, malic acid, lactic acid, citric acid, formic acid, benzoic acid, tartaric acid, carbonic acid, hydrochloric acid, sulfuric acid, nitric acid, nitrous acid, phosphoric acid, the like, or a combination thereof. In one embodiment, the acid is selected from the group consisting of ascorbic acid, citric acid, malic acid, hydrochloric acid, sulfuric acid, and any combinations thereof. In another embodiment, the acid can be citric acid. Alternatively, the acidifying agent can include an acidifying gas such as NO, N₂O, NO₂, CO₂, the like, or other acidifying gases. In one aspect, the acidifying gas may be NO. In another aspect, the acidifying agent can be an acidifying polymer or protein, such as alginic acid, an acidified gelatin, polyacrylic acid, and other acidifying polymers or proteins. In addition, acidifying agents may include compounds or molecules that produce or release an acid, including any of the aforementioned acids, upon addition to the NORS solution.

As described above, the amount of acidifying agent present in the solution can affect the rate of the reaction to produce NO. In one embodiment, the amount of acidifying agent is no greater than about 5.0% w/v of the solution. In another embodiment, the amount of acidifying agent is no greater than about 0.5% w/v. In another embodiment, the amount of acidifying agent is about 0.2% w/v. In a further embodiment, the amount of acidifying agent is about 0.07% w/v. In an additional embodiment, the amount of acidifying agent is about 0.07% w/v. In a further embodiment, the amount of acidifying agent is about 0.04% w/v. In yet another embodiment, the amount of acidifying agent is between about 0.07-5.0% w/v. In another embodiment, the amount of acidifying agent can be from about 2 mM to about 600 mM. In another embodiment, the amount of acidifying agent can be from about 5 mM to about 100 mM. In another embodiment, the amount of acidifying agent can be from about 5 mM to about 50 mM. In another embodiment, the amount of acidifying agent can be from about 100 mM to about 600 mM. It will be recognized that different acidifying agents can lower the NORS pH at different rates and to different degrees depending their specific properties and nature and suitable concentrations and concentration ranges of a given acidifying agent that are suitable for use as recited herein can be determined by one of ordinary skill in view of the present disclosure.

In one aspect, a therapeutically effective amount of a NORS can be from about 40 to about 10,000 ppm gNO. In one aspect, a therapeutically effective amount can be from about 40 to about 1000 ppm gNO. In one embodiment, the therapeutically effective concentration of gNO is from about 4 ppm to about 400 ppm gNO. In another aspect, the therapeutically effective amount of gNO can be from about 100 to about 220 ppm gNO. In another embodiment, the therapeutically effective concentration is from about 50 to about 200 ppm gNO. In a more specific aspect, the therapeutically effective amount can be about 160 ppm gNO. In another aspect, the therapeutically effective amount can be less than 160 ppm.

Without wishing to be bound by theory, it is believed that gNO can elicit an innate or natural immune response in a subject. Nitric oxide (NO) is a naturally occurring nano-molecule that is both a modulator and effector of the host innate immune response. Specifically, BRDc and other diseases or disorders can induce significantly increased expression of at least the pro-inflammatory cytokines IL-1β, TNF, and IL-8, as well as corresponding increases in IL-1β and TNF protein levels. Treatment with NORS can reduce the protein levels of IL-1β and TNF (63% and 42%, respectively). NORS treatment can also result in an increase in expression of toll-like receptor (TLR) proteins, which play a key role in the innate immune response, including facilitating host recognition of pathogens. For example, NORS treatment can increase expression of TLR3 (61%), TLR4 (44%), and TLR8 (45%). Hence, the nitroslyating agent NORS has the ability to provide protection against the development of BRDc and other diseases and disorders at least by limiting inflammation at the site of infection while simultaneously increasing TLR expression, enhancing the ability of the host to detect pathogens.

In one aspect, the adverse health condition can include at least one of a viral infection, a bacterial infection, a fungal infection, a parasitic infection, the like, or combinations thereof. In one aspect, the adverse health condition includes at least one of a viral infection and a bacterial infection, including clinical symptoms associated therewith. In one aspect, the adverse health condition includes clinical symptoms of Mannheimia haemolytica. In one aspect, the adverse health condition includes clinical symptoms associated with at least one of common cold, sinusitis, tonsillitis, pharyngitis, epiglottitis, laryngotracheitis, bronchitis, bronchiolitis, pneumonia, the flu (e.g. swine flu, avian flu, etc.), respiratory syncytial virus (RSV), tuberculosis, pertussis, enterovirus, severe acute respiratory syndrome (SARS), middle east respiratory syndrome (MERS), chronic obstructive pulmonary disease (COPD), and the like.

b. Biological Agent

In another embodiment of the invention, the immunomodulator composition includes a liquid NORS and at least one biological agent.

Suitable biological agents can include agents that are effective in preventing or treating infectious disease. Such biological agents can include immune enhancer proteins, immunogens, vaccines, antimicrobials, the like, or any combination thereof. Suitable immune enhancer proteins are those proteins known to enhance immunity. By way of a non-limiting example, a cytokine, which includes a family of proteins, is a known immunity enhancing protein family. Suitable immunogens are proteins which elicit a humoral and/or cellular immune response such that administration of the immunogen to a subject mounts an immunogen-specific immune response against the same or similar proteins that are encountered within the tissues of the subject. An immunogen may include a pathogenic antigen expressed by a bacterium, a virus, a parasite, or a fungus, for example. Preferred antigens include antigens which cause an infectious disease in a subject. According to the present invention, an immunogen may be any portion of a protein, naturally occurring or synthetically derived, which elicits a humoral and/or cellular immune response. As such, the size of an antigen or immunogen may be as small as about 5-12 amino acids and as large as a full length protein, including sizes in between. The antigen may be a multimer protein or fusion protein. The antigen may be purified peptide antigens derived from native or recombinant cells. The nucleic acid sequences of immune enhancer proteins and immunogens are operatively linked to a transcription control sequence, such that the immunogen is expressed in a tissue of a subject, thereby eliciting an immunogen-specific immune response in the subject, in addition to the non-specific immune response.

In another embodiment of the invention, the biological agent is a vaccine. The vaccine may include a live, infectious, viral, bacterial, or parasite vaccine or a killed, inactivated, viral, bacterial, or parasite vaccine. In one embodiment, one or more vaccines, live or killed viral vaccines, may be used in combination with the immunomodulator composition of the present invention. Suitable vaccines include those known in the art. Exemplary vaccines, without limitation, include adenovirus vaccine, coxsackie B vaccine, cytomegalovirus vaccine, dengue vaccine, Eastern equine encephalitis vaccine, ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine, Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine, West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, lyme disease vaccine, Staphylococcus aureus vaccine, Streptococcus pyogenes vaccine, syphilis vaccine, tularemia vaccine, Yersinia pestis vaccine, and other vaccines known in the art.

In yet another embodiment of the invention, the biological agent is an antimicrobial. Suitable antimicrobials include: quinolones, preferably fluoroquinolones, β-lactams, and macrolide-streptogramin-lincosamide (MLS) antibiotics.

Suitable quinolones include benofloxacin, binfloxacin, cinoxacin, ciprofloxacin, clinafloxacin, danofloxacin, difloxacin, enoxacin, enrofloxacin, fleroxacin, gemifloxacin, ibafloxacin, levofloxacin, lomefloxacin, marbofloxacin, moxifloxacin, norfloxacin, ofloxacin, orbifloxacin, pazufloxacin, pradofloxacin, perfloxacin, temafloxacin, tosufloxacin, sarafloxacin, gemifloxacin, and sparfloxacin. Preferred fluoroquinolones include ciprofloxacin, enrofloxacin, moxifloxacin, danofloxacin, and pradofloxacin. Suitable naphthyridones include nalidixic acid.

Suitable β-lactams include penicillins, such as benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin and clavulanic acid), azlocillin, carbenicillin, ticarcillin, mezlocillin, piperacillin; cephalosporins, such as cefalonium, cephalexin, cefazolin, cefapririn, cefquinome, ceftiofur, cephalothin, cefaclor, cefuroxime, cefamandole, defotetan, cefoxitin, ceftriaxone, cefotaxime, cefpodoxime, cefixime, ceftazidime, cefepime, cefpirome; carbapenems and penems such as imipenem, meropenem, ertapenem, faropenem, doripenem, monobactams such as aztreonam (Azactam), tigemonam, nocardicin A, tabtoxinine-B-lactam; and P-lactamase inhibitors such as clavulanic acid, tazobactam, and sulbactam.

Suitable MLS antibiotics include any macrolide, lincomycin, clindamycin, pirlimycin, erthyromycin, clarithromycin, roxithromycin, tilmicosin, gamithromycin, tulathromycin, etc.

Other antimicrobials include 2-pyridones, tetracyclines, sulfonamides, aminoglycosids, trimethoprim, dimetridazoles, erythromycin, framycetin, furazolidone, various pleuromutilins such as tiamulin, valnemulin, various, streptomycin, clopidol, salinomycin, monensin, halofuginone, narasin, robenidine, florfenicol, etc.

### Methods

a. Methods of Immune Stimulation

In one embodiment of the invention, a method of eliciting an immune response in a subject is described. The method can include administering to a subject a therapeutically effective amount of an immunomodulator composition. Such an immune response can be elicited in any suitable subject by administering a therapeutically effective amount of an immunomodulator composition to the subject. The therapeutically effective amount is an amount sufficient to elicit an immune response in the subject. The immunomodulator composition can include a liquid NORS.

In another embodiment of the invention, an immune response is elicited by administering a therapeutically effective amount of an immunomodulator composition, which includes a liquid NORS and a biological agent. It is contemplated that the biological agent may be mixed with or co-administered with the immunomodulator or administered independently thereof. Independent administration may be prior to or after administration of the immunomodulator. It is also contemplated that more than one administration of the immunomodulator and/or biological agent may be used to extend enhanced immunity. Furthermore, more than one biological agent may be co-administered with the immunomodulator, administered prior to the immunomodulator, administered after administration of the immunomodulator, or concurrently.

b. Diseases

The embodiments of the current technology can elicit an immune response in a subject such that the subject is protected from a disease that is amenable to elicitation of an immune response. As used herein, the phrase “protected from a disease” refers to reducing the symptoms of the disease; reducing the occurrence of the disease, reducing the clinical or pathologic severity of the disease, and/or reducing shedding of a pathogen causing a disease. Protecting a subject can refer to the ability of a therapeutic composition of the present invention, when administered to a subject, to prevent a disease from occurring, cure, and/or alleviate or reduce disease symptoms, clinical signs, pathology, or causes. As such, to protect a subject from a disease can include both preventing disease occurrence (prophylactic treatment) and treating a subject that has a disease (therapeutic treatment). In particular, protecting a subject from a disease can be accomplished by eliciting an immune response in a subject by inducing a beneficial or protective immune response which may, in some instances, additionally suppress, reduce, inhibit, or block an overactive or harmful immune response. The term “disease” refers to any deviation from the normal health of a subject and includes a state when disease symptoms are present, as well as conditions in which a deviation (e.g., infection, gene mutation, genetic defect, etc.) has occurred, but symptoms are not yet manifested.

Methods of the invention may be used for the prevention of disease, stimulation of effector cell immunity against disease, elimination of disease, alleviation of disease, and prevention of a secondary disease resulting from the occurrence of a primary disease.

The present invention may also improve the acquired immune response of the subject when co-administered with a vaccine versus administration of the vaccine by itself. Generally a vaccine once administered does not immediately protect the subject as it takes time to stimulate acquired immunity. The term “improve” refers, in the present invention, to elicitation of an innate immune response in the subject until the vaccine starts to protect the subject and/or to prolong the period of protection, via acquired immunity, given by the vaccine.

Methods of the invention include administering the composition to protect against infection of a wide variety of pathogens. The composition administered may or may not include a specific antigen to elicit a specific response. It is contemplated that the methods of the invention will protect the recipient subject from disease resulting from infectious microbial agents including, without limitation, viruses, bacteria, fungi, and parasites. Exemplary viral infectious diseases, without limitation, include those resulting from infection with rhinoviruses, influenza viruses, respiratory syncytial virus (RSV), molluscum contagiousum, herpes simplex virus-1, herpes simplex virus-2, human herpesvirus 6, human herpesvirus 7, varicella-zoster virus, hepatitis A, norovirus, rotavirus, Epstein-Barr virus, west nile virus, junin virus, astrovirus, polyomaviruses, machupo virus, sabia virus, sapoviruses, alphavirus, coronaviruses, dengue viruses, cytomegalovirus, ebolavirus, parvovirus, hantavirus, heartland virus, hepatitis A virus, hepatitis B virus, hepatitis C virus, hepatitis D virus, hepatitis E virus, human bocavirus, human metapneumovirus, human papillomavirus, lassa virus, mumps virus, measles virus, Marburg virus, monkeypox virus, chicken pox virus, poliovirus, rabies virus, rubella virus, yellow fever virus, recombinants thereof, the like, and other viruses known in the art. Exemplary bacterial infections, without limitation, include those resulting from infection with gram positive or negative bacteria and Mycobacteria such as Escherichia coli, Clostridium perfringens, Clostridium difficile, Campylobacter jejuni, Clostridium botulinum, Clostridium tetani, Ureaplasma urealyticum, Mycoplasma pneumoniae, Leptospira interrogans, Leptospira santarosai, Leptospira weilii, Leptospira noguchi, Bacillus anthracis, Bacillus cereus, Treponema pallidum, Corynebacterium diphtheriae, Mycobacterium tuberculosis, Mycobacterium leprae, Mycobacterium ulcerans, Bartonella henselae, Bartonella quintana, Bordetella pertussis, Borrelia burgdorferi, Borrelia garinii, Borrelia afzelii, Borrelia recurrentis, Brucella abortus, Brucella canis, Brucell melitensis, Brucella suis, Chlamydia pneumonia, Chlamydia trachomatis, Chlamydophila psittaci, Enterococcus faecalis, Enterococcus faecium, Francisella tularensis, Haemophilus influenza, Heliobacter pylori, Legionella pneumophila, Listeria monocytogenes, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyrogenes, Treponema pallidum, Vibrio cholerae, Yersinia pestes, Yersinia enterocolitica, Yersinia pseudotuberculosis, other bacteria known in the art, or a combination thereof. Exemplary fungi or mold infection, without limitation, include those resulting from infection with Candida albicans, Candida glabrata, Candida rugosa, Candida parapsilosis, Candida tropicalis, Candida dubliniensis, other Candida species, Aspergillus fumigatus, Aspergillus flavus, Aspergillus clavatus, other Aspergillus species, Crytpococcus neoformans, Crytococcus laurentii, Crypotcoccus albidus, Crytococcus gattii, other Cryptococcus species, Histoplasma capsulatum, Pneumocystis jirovecii, Stachybotrys chartarum, other infectious fungi or mold known in the art, or a combination thereof. Exemplary parasites include, without limitation, Acanthamoeba spp., Balamuthia mandrillaris, Balantidium coli, Blastocystis spp., Cryptospordium spp., Cyclospora cayetanensis, Dientamoeba fragilis, Entamoeba histolytica, Giardia lamblia, Isospora belli, Leishmania spp., Naegleria fowleri, Plasmodium falciparum, Plasmodium vivax, Plasmodium ovale curtisi, Plasmodium ovale wallikeri, Plasmodium malariae, Plasmodium knowlesi, Rhinosporidium seeberi, Sarcosystis bovihominis, Sarcocystis suihominis, Toxoplasma gondii, Trichomonas vaginalis, Trypanosoma brucei, Trypanosoma cruzi, other parasites known in the art, or a combination thereof.

c. Subjects

The methods of the invention may be administered to any suitable subject, including a human, primate, bovine, goat, swine, canine, feline, equine, bison, alpaca, llama, sheep, or the like. In some specific examples, the subject can be a human.

d. Administration

A variety of administration routes are available. The particular mode selected will depend, of course, upon the particular biological agents selected, the age and general health status of the subject, the particular condition being treated and the dosage required for therapeutic efficacy. The methods of this invention may be practiced using any mode of administration that produces effective levels of an immune response without causing clinically unacceptable adverse effects. The compositions may conveniently be presented in unit dosage form and may be prepared by any of the methods well known in the art.

Vaccination can be performed at any suitable age. The vaccine may be administered intravenously, intramuscularly, intradermal, intraperitoneal, subcutaneously, by spray/aerosol, orally, intraocularly, intratracheally, intranasal, or by other methods known in the art. Further, it is contemplated that the methods of the invention may be used based on routine vaccination schedules.

In some examples, the immunomodulator may be administered to the subject as an extended release formulation of gNO, and optionally with a carrier formulation, such as microspheres, microcapsules, liposomes, etc. The immunomodulator can be administered topically or internally, locally or systemically, to treat a microbial (e.g. viral, bacterial, fungal, parasitic, etc.) disease or disorder. Additionally, the immunomodulator can be administered as a liquid, a spray, a vapor, micro-droplets, mist, footbath, the like, or any other form that provides the desired release of gNO from the immunomodulator, or a combination thereof.

A variety of administration volumes can be used, depending on the mode of administration and the target treatment area. For example, in some cases, a treatment volume of from about 0.1 ml to about 5000 ml, or a treatment volume of from about 0.5 ml to about 500 ml can be used. In some specific examples, an immunomodulator composition that releases a therapeutically effective amount of gNO can be deposited in, on, or around the subject's nose in an amount from about 0.1 ml to 50 ml. In another aspect, an immunomodulator that releases a therapeutically effective amount of gNO can be deposited in, on, or around the subject's nose in an amount from about 0.5 ml to about 20 ml. In a more specific aspect, an immunomodulator that releases a therapeutically effective amount of gNO can be deposited in, on, or around the subject's nose in an amount of about 1 ml to about 5 ml or about 10 ml. Such amounts can be made through a single administration or an administration event that includes multiple administrations.

Alternatively, an immunomodulator composition that releases a therapeutically effective amount of gNO can be administered to the nares and/or mouth of the subject via a pneumatic channel fluidly connected to a NORS reservoir. In another aspect, an immunomodulator composition that releases a therapeutically effective amount of gNO can be administered by sufficiently approximating the subject to a NORS reservoir such that a therapeutically effective amount of gNO is delivered to the nares and/or mouth of the subject.

The immunomodulator may also be administered by providing a nitric oxide releasing compound or agent and an acidifying compound or agent. The nitric oxide releasing agent and the acidifying agent can be combined to provide an activated immunomodulator. Any suitable nitric oxide releasing compounds and acidifying compounds can be used, as described herein. Strong acidifying agents can cause rapid production and release of gNO. Weaker acidifying agents can produce a prolonged production and release of gNO. Careful control of the amounts and combinations of acidifying agents incorporated in the immunomodulator can prolong the release of a therapeutically effective amount of gNO, thus allowing the immunomodulator to be prepared well in advance of administration. However, this is not always a desirable scenario. In some cases, it can be desired to produce a strong and sudden burst of gNO at the site of the disease, disorder, or condition being treated, and thus combining the nitric oxide releasing compound and the acidifying agent just prior to administration or during administration, or at the treatment site can be preferred. Alternatively, it may be desirable to administer either the acidifying agent or the nitric oxide releasing agent to the subject and subsequently activate it by administration of the corresponding nitric oxide releasing agent or acidifying agent. Hence, the immunomodulator can be administered to a subject before, during, or after activation of the immunomodulator. In one aspect, the immunomodulator can be activated up to 24 hours before administration. In one aspect, the immunomodulator can be activated up to 8 hours before administration. In one aspect, the immunomodulator can be activated up to 1 hour before administration. In one aspect, the immunomodulator can be activated up to 30 minutes before administration. In one aspect, the immunomodulator can be activated up to 10 minutes before administration. In one aspect, the immunomodulator can be activated up to 5 minutes before administration. In one aspect, the immunomodulator can be activated up to 1 minute before administration. In another aspect, the immunomodulator can be activated during administration. In another aspect, the immunomodulator can be activated after administration.

As previously noted, the immunomodulator can provide an extended release of gNO to a subject in need thereof. By “extended release,” it is meant that a therapeutically effective amount of gNO is released from the formulation at a controlled rate for a specified duration such that therapeutically beneficial levels (but below toxic levels) of the component are maintained over an extended period of time following immunomodulator administration. Thus for example, release can occur about 5 seconds to about 24 hours, thus, providing, for example, a 30 to 60 minute, or several hour, dosage form. In one embodiment, the NO gas is released over a period of at least 30 minutes. In another embodiment, the NO gas is released over a period of at least 8 hours. In another embodiment, the NO gas is released over a period of at least 12 hours. In another embodiment, the NO gas is released over a period of at least 24 hours. An extended release NORS is beneficial in that the solution can be administered to the subject over a short period of time, while the release of NO from the solution continues following administration. Moreover, the use of an extended release immunomodulator allows the subject to remain ambulatory following administration of the solution, as opposed to remaining stationary while being connected to a NO-releasing device in order to receive treatment.

The duration of administering the immunomodulator to the subject may be varied in order to optimize delivery. In one embodiment, the immunomodulator is administered to the subject over a time period of about 1 second or less. In another embodiment, administration time can be about 5 seconds or less. In another embodiment, administration time can be about 5 seconds. In another embodiment, the administration time can be about 30 seconds or less. In another embodiment, the administration time can be about 1 minute or less. In another embodiment, the administration time can be about 2 minutes or less. In another embodiment, the administration time can be about 10 minutes or less. In another embodiment, the administration time can be about 30 minutes or less.

In one embodiment, the immunomodulator is administered by itself to the subject prior to challenge (or infection). In another embodiment, the immunomodulator is administered by itself to the subject post challenge (or infection). In yet another embodiment, the immunomodulator is administered by itself to the subject at the same time as challenge (or infection). In a further embodiment, the immunomodulator composition is co-administered at the same time as the vaccination prior to challenge. In yet a further embodiment, the immunomodulator composition is co-administered at the same time as the vaccination at the same time as challenge (or infection). In another embodiment, the immunomodulator composition is administered prior to vaccination and challenge. In a further embodiment, the immunomodulator composition is administered after vaccination but prior to challenge. In a further embodiment, the immunomodulator composition is administered after challenge to a subject that has been vaccinated prior to challenge (or infection).

In one embodiment, the immunomodulator is administered from about 1 to about 14 days prior to challenge or from about 1 to about 14 days post challenge. In another embodiment, the immunomodulator is administered from about 1 to about 7 days prior to challenge or from about 1 to about 7 days post challenge. In yet another embodiment, the immunomodulator is administered 1, 2, 3, 4, 5, 6, 7 days prior to challenge or 1, 2, 3, 4, 5, 6, 7 days post challenge.

In another embodiment, a method of improving the acquired immune response of a subject is described. The method includes administering to the subject a therapeutically effective amount of a NORS. As previously described, NO is a naturally occurring nano-molecule that is both a modulator and effector of the host innate immune response. Treatment with NORS can reduce the levels of pro-inflammatory proteins or cytokines such as IL-1β, IL-8, IL-10, and TNF. In some examples, NORS treatment can reduce a level of one or more pro-inflammatory proteins or cytokines by at least 30%, at least 50%, at least 70%, or more in a subject as compared to an untreated subject or as compared the treated subject prior to or without receiving a NORS treatment. This can be accomplished at a target location, such as a treatment area or situs of administration, within a predetermined period (such as 4 hours, 8 hours, 12 hours, 20 hours, or 24 hours). Further, NORS treatment can increase the expression of TLRs, such as TLR3, TLR4, and TLR8. In some examples, NORS treatment can increase the expression of one or more TLRs by at least 30%, 50%, 70%, or more in a subject as compared to an untreated subject or as compared the treated subject prior to or without receiving a NORS treatment. This can also be accomplished at a target location, such as a treatment area or situs of administration, within a predetermined period (such as 4 hours, 8 hours, 12 hours, 20 hours, or 24 hours). In some examples, treatment with NORS can also reduce a period of fever by 1 day, 2 days, 3 days, or more in a subject as compared to an untreated subject or the treated subject prior to or without NORS administration. In some examples, the reduced period of fever can also reduce weight loss in the subject. These are just some of the immumodulating effects that result from administration of a therapeutically effective amount of a NORS.

As various changes could be made in the above composition, products and methods without departing from the scope of the invention, it is intended that all matter contained in the above description and in the examples given below, shall be interpreted as illustrative and not in a limiting sense.

### EXAMPLES

### Example 1

Eighty-five, crossbred, multiple sourced, commingled commercial weaned beef calves were obtain for these studies. All studies were conducted at the Lacombe Research Centre beef research facility and all management practices followed Canadian Council of Animal Care guidelines and Canadian Beef Cattle Code of Practice guidelines. In addition, the research protocols were reviewed and approved by the Lacombe Research Centre animal care committee. The calves were procured through a conventional auction system and all animals had been exposed to between 4-6 h of transport prior to the study. These calves were chosen in order to provide study groups displaying a bovine respiratory disease (BRDc) incidence range of 30-60% which is typical of the beef industry in Canada for these “put together” herds of cattle. On arrival at Lacombe the calves were off loaded, weighed, and sampled for saliva and blood using methods known in the art.

The calves were randomized to treatment and control groups, labeled with color coded ear tags and numbers. NORS was delivered with a spray device. This solution was tested and verified to release 160 ppm NO in a 3 l/m flow of medical air (Praxair, Cananda), for 30 min. In brief, 32 ml of the solution was sprayed into a two inch diameter vinyl chloride tube and inserted into environmentally controlled system where NO was measured using chemiluminescence (Sievers Nitric Oxide Analyzer NOA 280i). Animals were restrained in a conventional hydraulic cattle-handling catch and given either a placebo (saline) or treatment (NO) by an individual blinded as to the intervention. Each animal received 1 spray (8 ml), alternating into each nostril, twice, for a total of 32 ml before being released into the feeding lot pen areas. The duration of treatment administration was less than 5 s.

Animals were then placed into outdoor pens measuring approximately 60×60 m and were bunk feed ad libitum a balanced cereal silage diet, which met or exceeded National Research Council recommendations. The animals also had free access to water and were provided a straw bedding area with a roof covering.

While contained in their receiving pens the calves were monitored daily by trained personnel, whom were blinded as to the treatment interventions, for clinical signs of illness. Briefly, clinical scores were designed to identify BRDc and were based on the appearance of four criteria as follows:

Respiratory insult: (0-5): 0=no insult, normal breath sounds (NBS); 1=Very Fine Crackle (rale) (VFCR) on auscultation and/or a moderate cough; 2=Fine Crackle (subcrepitant) (FCR) on auscultation and/or a moderate nasal discharge and moderate cough; 3=Medium Crackle (crepitant) (MCR) on auscultation and/or a moderate to severe viscous nasal discharge with cough; 4=Course Crackles (CCR), tachypnoea (>15% of the norm) and/or a severe nasal discharge with respiratory distress and obtunded lung sounds and 5=CCR with dyspnoea, tachypnoea, marked respiratory distress and/or lung consolidation.

Digestive insult: (0-5): 0=no insult, normal, eating and drinking; 1=mild or slight diarrhoea with slight dehydration (<5%) and reduced eating; 2=moderate diarrhoea with 10% dehydration and reduced feed intake (<50%); 3=moderate to severe diarrhoea with 10% or less of feed intake and more than 10% dehydration; 4=severe diarrhoea, and less than 10% of normal feed intake and 5=severe diarrhea and not eating, not drinking and dehydrated.

Temperature score: Core temperature (rectal) (0-5): 0=<37.7° C.; 1=37.7-38.2° C.; 2=38.3-38.8° C.; 3=38.9-39.4° C.; 4=39.5-40.0° C. and 5=>40° C. Rectal or core temperatures for the calves were collected at the start and end of the study only as these were the times that the animals were restrained.

Disposition or lethargy score: (0-5): 0=no lethargy, normal posture; 1=mild anorexia or listlessness, depressed appearance; 2=moderate lethargy and depression, slow to rise, anorectic; 3=recumbent or abnormal posture, largely depressed; 4=prostrate, recumbent or abnormal posture and 5=death.

Animals displaying overt clinical symptoms of BRDc as identified by a blinded pen keeper were rescued and subsequently received immediate treatment as recommended by the Lacombe Research Centre veterinarian followed by continued monitoring and re-treatment if required. These animals were classified as true positive (TP) in the statistical analysis.

The determination of an animal true positive or negative for BRDc was based on the comparison to a set of “gold standard” values as known in the art. This approach is commonly promoted in both veterinary and human medical diagnostic studies. In the current study, the criteria for a true positive animal for BRDc was defined as an animal displaying three or more of the following signs; a core temperature of >40° C. (or 103.5° F.), a white blood cell count of less than 7 or greater than 11×1000/1 L, a clinical score of >3 or a neutrophil/lymphocyte ratio of <0.1 (leucopaenia) or >0.8 (neutrophilia). A true negative animal was defined as an animal displaying a score of 0 or 1.

Salivary and serum cortisol levels were analyzed using an enzymatic assay known in the art. Hematology values were measured on a Cell-Dyn 700 Hematology Analyser (Sequoia-Turner Corp. Mountain View, Calif.). Differential blood cell counts were determined utilizing stained blood smears (Geisma-Wright quick stain) and direct microscope examination of 100 cells. For laboratory assessments, all calves were monitored at the beginning of the study and again three to four weeks later.

The results were analyzed using the unpaired Student's t-test for comparison between any two groups. Group means were statistically tested by least squares means (two-tailed t-test). Data analysis and graphical presentation were done using a commercial statistics package (Graphpad-Prism V 3.0, GraphPad Software Inc., USA). Unless otherwise specified, p<0.05 indicated statistical significance. Results were reported as the mean±standard deviation.

Three different studies were done. Eighty-six multi-sourced, commingled commercial weaned beef calves were enrolled in the study and randomized into either treatment or control cohort. When analyzing the results, animals that arrived to the feedlot as TP, were discarded from the analysis which left 40 control animals and 42 in the treatment group. As can be seen in Table 1, the remaining animal cohorts were not significantly different in any of the parameters tested. No significant difference was found between average weight (p=0.81) of the two groups with values of 287.7 kg (SD 37.8) for control and 290.9 kg (SD 46.8) for treatment. All baseline blood work including total white blood cells and specifically neutrophils, lymphocytes, monocytes, eosinophils and basophils were not significantly different between the two cohorts. Three animals in the control group and none in the treatment group were identified by the pen keeper using normal commercial criteria and were rescued with conventional antibiotics and categorized as treatment failures for statistical analysis.

TABLE 1
Demographics-the average value for treatment or control groups for
weight, temperature and all blood parameters that were tested.

| | Weight | Temp F | Wbc | Neut | % Neut | Lymph | % Lymph | Mono | % Mono | Eos | % Eos | Baso | % Baso | Rbc | Hgb | Hctm |
|---|---|---|---|---|---|---|---|---|---|---|---|---|---|---|---|---|
| C avg | 633 | 103.0 | 8.3 | 1.1 | 13.6 | 5.4 | 65.1 | 0.8 | 10.5 | 1.0 | 10.1 | 0.1 | 0.7 | 9.8 | 13.3 | 39.1 |
| C std | 83 | 1.1 | 1.9 | 0.9 | 9.3 | 1.7 | 12.5 | 0.3 | 4.3 | 0.9 | 7.4 | 0.0 | 0.3 | 1.2 | 1.2 | 3.2 |
| Tx avg | 640 | 102.9 | 8.3 | 1.1 | 13.7 | 5.4 | 64.5 | 0.9 | 11.0 | 0.8 | 9.7 | 0.1 | 1.1 | 9.6 | 13.2 | 38.5 |
| Tx std | 104 | 0.9 | 1.6 | 0.9 | 10.3 | 1.7 | 14.4 | 0.5 | 6.5 | 0.7 | 8.1 | 0.1 | 2.2 | 1.2 | 1.3 | 3.8 |
| T test | 0.8 | 0.6 | 1.0 | 1.0 | 1.0 | 1.0 | 0.8 | 0.6 | 0.7 | 0.4 | 0.8 | 0.3 | 0.3 | 0.5 | 0.7 | 0.5 |

All animals tolerated the nitric oxide treatments well. Some of the animals sneezed but none exhibited coughing or other clinical signs of distress. However, behavioral differences in the tolerance of treatment between the cohorts were not quantified. There were no adverse events nor serious adverse events observed in either cohort. No animals died during the time of the study. Mean salivary and cortisol levels were equivalent in each group (Control 5.4±5.7 nmol/L; Treatment 6.66±5.5 nmol/L) without significant differences (p=0.09).

As can be seen in Table 2, during days 1-14, 13 animals from the control group and 5 animals from the treatment group were identified as TP. The table shows values recorded for all 4 parameters determining TP/TN for all TP animals. Temperature, clinical score, white blood count, neutrophil/lymphocyte ratio were also included. All sick animals had 3 or 4 parameters recorded below or above the defining value for TP. This scoring approach provides a more robust definition of sick animals as compared to looking at just a temperature threshold alone. All animals had clinical scores above 3 and 15 out the 18 animals had temperature recorded as 103.5° F. or higher. Thirteen out of the 18 TP animals were also recognized by the pen keeper as sick.

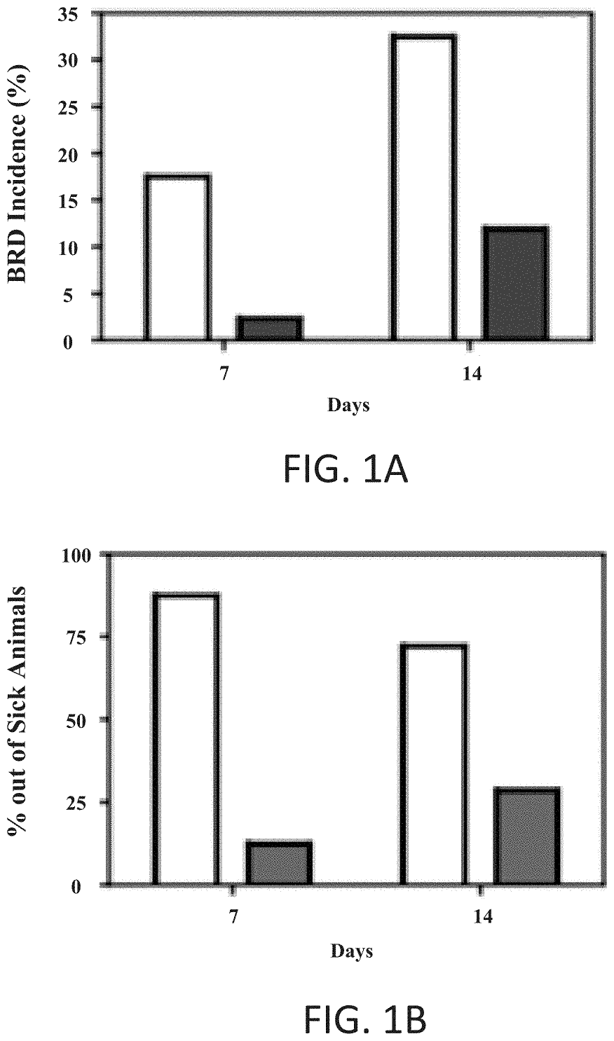

In terms of a BRDc incidence in this model, of these 82 calves evaluated, after 7 days post arrival, 8 displayed true positive for BRDc (10%). As shown in FIG. 1a, 7 animals (17.5%) out of the 40 in the control group and 1 (2.4%) out of 42 in the NO treated group were identified as TP in the first week. Another way to look at these data (FIG. 1b) is that of these 8 animals, one (12.5%) was from the NO treated group and seven (87.5%) were from the saline control group. This represents a very significant reduction of the incidence of BRDc between the treatment and control cohorts with a single NORS treatment upon arrival into the stockyard (p<0.001). During the first 14 days, 18 animals (22%) had an incidence of BRDc and of these 13 (72.2%) were in the control group whereas only 5 (27.8%) were in the treatment cohort (FIG. 1b).

TABLE 3
Day of sickness. Table shows the day in which an animal was recorded
sick, post arrival to feedlot.

| | | Day 0 | Day 1-7 | Day 8-10 | Day 11-14 | Day 15-28 |
|---|---|---|---|---|---|---|
| Control | # New TP | 3 | 7 | 3 | 3 | 0 |
| | Cumulative sick (from day 1) | | 7 | 10 | 13 | 13 |
| | Remaining | 40 | 33 | 30 | 27 | 27 |
| Tx | # New TP | 2 | 1 | 2 | 2 | 6 |
| | Cumulative sick (from day 1) | | 1 | 3 | 5 | 11 |
| | Remaining | 42 | 41 | 39 | 37 | 31 |

Table 3 shows new sick animals per time period, defined at either testing day or when animals were pulled out. Animals were pulled out of the study and deemed clinically sick when the animal herdsman determined that the animal was deemed sick by normal commercial feedlot assessment. Looking at the first 2 weeks after treatment, 13 (32.5% of total control group) animals out of the control group had a TP score, while only 5 (11.9% of total Tx group) had a TP score out of the treatment group. It should be noted as well that 3 more animals out of the control group (and none of the treatment) were pulled out during these 2 weeks although they were borderline and did not turn out to be TP. These animals as having a TP incidence of BRDc in the above analysis were included in the results. The median day that an animal became sick, after arriving at the feedlot, was 8 days in the control compared to 18 days in the treatment group. When looking at 15-28 days post treatment, there was no effect seen. Further, when looking at the cumulative number of sick animals from day 1 to day 28, 33% of the control group and 26% of the treatment group were identified as TP.

These data, collected from three separate randomized and blinded studies performed in a conventional feedlot, show that NO significantly decreased the incidence of BRDc, as defined by true positive rigor, by a difference of 75% as compared to a saline placebo (87.5% of sick animals were from control vs 12.5% from treatment group). This test used a naturally occurring BRDc model from multi-sourced co-mingled animals acquired from a commercial auction and transferred to a research feedlot. Further, duration of effectiveness for NORS treatment was up to 14 days which is similar to most antibiotics.

Additionally, once an animal was treated with NO, if it did become sick, the illness was delayed. The average day of an animal from treatment group to get sick was 18 days post arrival to the research feedlot while it was 8 days for the control group. Reduction and delayed onset of BRDc observed in this study is likely to be related to nitric oxide released from NORS. Release of NO from NORS was verified, prior to the study, in a bench test model where a continuous flow of air over NORS resulted in 160 ppm nitric oxide.

These results are similar to those reported in metaphylactic use of antibiotics to treat BRDc in clinical trials. Metaphylactic use of antibiotics has been shown to reduce and delay the incidence of BRDc as defined by an undifferentiated fever of greater than 104° F. (41.7° C.) in beef cattle entering feedlots. Cattle presenting with undifferentiated fever treated with metaphylactic antibiotics have lower incidence of mortality, a higher weight and quality of meat when they are dressed.

Unlike antibiotics, requiring pre-slaughter withdrawal periods (as determined by the FDA), nitric oxide is unlikely to have any residue in the meat product, due to its short half-life. Moreover, antibiotics and NO have different mechanisms of actions; antibiotics are classified based on specific targets whereas gNO possesses a wide-ranging antimicrobial targets that are essential to the basic biochemistry of the microbes. Recent studies in bacteria have suggested that NO has an affinity for reduced surface thiols and divalent metal centres in intracellular enzymes. It is predicted that nitric oxide will attach to surface cysteines causing the formation of S-nitrosylation (—SNO) sites, which perturb enzyme structure and/or catalytic activity. Another mechanism is the reaction of NO with oxygen or superoxide to spontaneously produce reactive nitrogen and oxygen intermediates, resulting in the formation of multiple antimicrobial intermediates. These reactive nitrogen oxide species cause oxidative and nitrosative damage by altering DNA, inhibiting enzyme function, and inducing lipid peroxidation, which account for the majority of NO cytotoxic effects. As a result, nitric oxide seems to be effective against a wide spectrum of bacteria, viruses and fungi while antibiotics are specific to bacteria. Therefore, the potential added advantage of NO, over antibiotics, is its anti-viral effect. NO may ameliorate the pathogenesis of BRDc by lowering the viral load and thereby reducing susceptibility of the animal to bacterial infection.

A major concern in food producing animals is the emergence of drug resistant bacteria. Nitric oxide production and use as the first line of defense in the immune system has been preserved genetically across many species. Since NO is a broad non-specific antimicrobial, rendered by its multiple intracellular biochemical targets, the risk of developing resistance to NO is likely to be ameliorated. It is postulated that the microbicidal activity of the NO released from NORS is fundamental to the biochemistry of both bacteria and viruses that survivors are unlikely to induce microbes to become “drug resistant”. This is evidenced by the lack of reported resistant bacteria/viruses in the human infant population that has been receiving suboptimal “antimicrobial” doses over the last decade.

### Example 2

### BHV-1 Viral Preparation.

The BHV-1 viral strain (clinical isolate) was obtained from the Animal Health Center (British Columbia ministry of agriculture) in Abbotsford, Canada. A Stock of the BHV-1 virus was grown in Madin-Darby Bovine Kidney Epithelial (MDBK) cells (ATCC® CCL-22™) for 48 hrs, with medium containing 2% fetal bovine serum (FBS). The stock of virus was prepared as clarified cell-free supernatants. Virus concentration of the stock was determined by standard plaque assay on MDBK cells. The BHV-1 stock virus titer was calculated to be 1×10⁷ to 4.4×10⁷ plaque forming units (PFU)/ml. Aliquots (1 ml) of viral stock were stored at −80° C. A fresh aliquot of stock was thawed and used for each experiment.

### LPS Bacterial Extraction Preparation.

M. haemolytica bacterial cultures were isolated and obtained from the Agriculture and Agri-Food Canada Research Centre (Lethbridge, Canada). Bacteria were grown to 0.5 McFarland standard. Subsequent 0.5 ml aliquots of these preparations containing approximately 2.5×10⁸ cfu/ml were mixed with 0.5 ml 50% sterilized glycerol and stored at −80° C. When needed, 100 μl of the freshly defrosted solution was added to 3 ml of Brain-Heart Infusion (BHI) broth (Becton, Dickinson and Company, Franklin Lakes, N.J., United States) and placed in a 37° C. shaker for 24 hrs. Approximately 0.5 ml of the newly grown bacteria was then added to 24.5 ml of BHI broth and placed in a 37° C. shaker for 24 hrs. The LPS fraction was then isolated and purified using the LPS Extraction kit as per the manufactures' instructions (iNtRON Biotechnology Inc., Seoul, South Korea). The isolated LPS was then aliquoted into vials (30 μl each) at a concentration of 1 μg/μl and stored at −20° C.

### Blood Collection and PBMC Isolation for In Vitro Study.

Peripheral blood was collected into lithium heparin tubes from multiple healthy male Holstein-Friesian cattle aged between 6 and 12 months on five separate dates (located at the University of British Columbia's Dairy Research and Education Center, Agassiz, Canada). The blood was transported on ice and pooled prior to PBMC isolation. Approximately 250 ml of blood was divided between ten 50 ml conical tubes with 25 ml of blood being layered onto 20 ml of Histopaque®-1083 (Sigma-Aldrich, St. Louis, Mo., USA). The tubes were centrifuged for 30 minutes at 805 g with the breaks not applied. The PBMC layer was collected from each tube, combined into four, 50 ml conical tubes and washed with 40 ml of PBS (—MgCl,—CaCl) at 201 g for 10 min. The pellet from each tube was re-suspended in 5 ml of PBS (—MgCl,—CaCl) and layered onto 5 ml of Histopaque®-1083 and centrifuged under the same conditions as before. The PBMC layer was again collected from each tube and washed twice with 40 ml of PBS (—MgCl, —CaCl) at 201 g. Following this, the pellets were combined and re-suspended in 30 ml RPMI 1640 media (Sigma-Aldrich) containing L-glutamine and antibiotics, supplemented with 3% FBS and incubated for 16 hrs (in a 75-cm² cell culture flask) at 37° C., 5% CO₂.

### Preparation of NORS.

Nitric oxide releasing solution (NORS) was prepared by diluting 1.0 M sodium nitrite in saline, to a working concentration of 22.5 mM, using 0.9% saline. The pH of the solution was subsequently lowered to a pH of 3.5 using citric acid. The NORS solution was then filter sterilized into 1.5 ml tubes. NORS was prepared immediately prior to use for each experiment.

### Establishment of In Vitro BRDc Infection Model and NORS Challenge Treatment.

### Viral Infection.

Following incubation to acclimatize, the bovine PBMCs were counted using a hemacytometer. Cells were further diluted with media to reach a concentration of 3×10⁶ cells per ml. The cells were then divided into two, 75-cm² cell culture flasks. The cells in one of these flasks were inoculated with BHV-1 at a multiplicity of infection (MOI) of 0.001 while the other was used as a control. Both flasks were then incubated at 37° C. for 1 hr and manually swirled every 5 min to establish the infection within the cells prior to NORS treatment. The NORS treatment was delivered post BHV-1 infection, but prior to cell culturing with LPS, to best reflect treatment delivery timing in feedlots.

### NORS Treatment.

Directly following the BHV-1 infection, the cells were counted again and re-suspended in RPMI 1640 media without FBS, at a cell concentration of 2.9×10⁶ cells per 30 μl. The two cell mixtures (infected and control) were placed in separate 1.5 ml vials. Thirty μl of the BHV-1 infected cells were then aliquoted into six, 1.5 ml tubes (two sets of three experimental replicates). Immediately, 100 μl of NORS was added to each tube, then capped and incubated for 2.5 min at room temperature (RT). Following incubation, 1 ml of RPMI with 3% FBS was added to each tube to increase the pH of the mixture above 6 in order to neutralize the production of NO (in some examples NO can be more significantly released from NORS at low pH). The contents of each tube were then added to a well on a 12 well culture plate (Greiner Bio-One North America Inc., Monroe, N.C., United States). The same procedure was repeated using the non-infected control cells. This process was repeated using sterile saline at a pH of 5.5 (Baxter International Inc., Deerfield, Ill., Untied States), instead of the NORS, to act as a treatment control. Each procedure was carried out in duplicate to provide two time points. The plates were then placed back in the 37° C. incubator for 24 hrs. LPS culturing was performed 24 hrs post NORS treatment to prevent over stimulation of the cells.

### LPS Culture and Sample Harvesting.

Following 24 hrs incubation at 37° C., the culture plates were removed and 100 ng (50 μl of 2 ng/μl) of LPS extracted from M. haemolytica was added to 3 of the wells containing BHV-1 infected cells and to 3 of the wells containing non-infected control cells, on each culture plate, for a final LPS concentration of approximately 100 ng/ml per well. This resulted in a final plate layout with three wells each of (1) BHV-1 infected PBMCs, (2) BHV-1 infected PBMCs cultured with LPS, (3) non-infected PBMCs cultured with LPS and (4) non-infected cell control PBMCs. The plates were gently shaken for 1 hr at RT to ensure uniform mixing and then incubated at 37° C. Four hrs (T1) after the initiation of the LPS culture, a treatment and control plate were removed from the incubator. The culture supernatant from each well was pipetted into 1.5 ml tubes and centrifuged at 453 g for 5 minutes to pelletize any cells present. The supernatants were then pipetted into fresh 1.5 ml tubes for protein assay work and placed at −20° C. for later analysis. On removal of the culture supernatant from the plate wells, 0.5 ml of RNAprotect® Cell Reagent was added into each well in order to stabilize the RNA in the cultured cells. Following 10 min of incubation at RT, the reagent was pipetted up and down repeatedly to help detach the cells from the culture plate. The mixtures from all three experimental replicates were then combined at this stage and pipetted into a 2 ml tube while the pellets in the centrifuged tubes were re-suspended using 300 μl of the combined mixture and pipetted into the 2 ml tube. These samples were then processed for RNA extraction within 2 hrs. Twenty hrs (T2) after the beginning of the LPS culture, the second set of plates were processed using the same methodology as previously described. The time points at which the mRNA and protein responses were measured were selected to capture an early response post LPS culturing and a second more established response.

### N.B.

T1—28 hrs post NORS, 30 hrs post BHV-1 infection, 4 hrs post LPS culturing; T2—44 hrs post NORS, 46 hrs post BHV-1 infection, 20 hrs post LPS culturing.

### RNA Extraction and Purification.

Total RNA was extracted from the PBMC samples stabilized in RNAprotect® Cell Reagent using the RNeasy Plus Mini Kit, together with the QIAshreddar, as per the manufacturer's instructions (Qiagen, Venlo, Limberg, Netherlands). RNA quality was assessed using the 18S/28S ratio and RNA integrity number (RIN) (all sample's RIN value >7.5) on an Agilent 2100 bioanalyzer with a RNA 6000 Nano LabChip kit (Agilent Technologies, CA, USA) while RNA quantities were determined using the NanoDrop® (NanoDrop Technologies, Inc., Wilmington, Del., USA). cDNA synthesis and RT-qPCR.

Five hundred ng of total RNA from each sample was reverse-transcribed into cDNA using a MultiScribe™ Reverse Transcriptase, as part of the High-Capacity cDNA Reverse transcription Kit with RNAse inhibitor according to the manufacturer's instructions (Applied Biosystems, Foster City, Calif., USA). Bos taurus gene sequences obtained from the NCBI GenBank database were used to design oligonucleotide primers for candidate genes using the Primer3 software package. Sequence specificity was confirmed for each primer set using the NCBI BLAST tool. Where possible, primers for RT-qPCR were designed to span an intron and were commercially synthesized (Eurofins MWG Operon LLC, Huntsville, Ala., USA). Details for gene-specific primer sets are shown in Table 4. Each RT-qPCR reaction was performed in duplicate with a total volume of 20 μl which consisted of 5 μl of cDNA (0.5 ng/μl), 10 μl of Fast SYBR Green Master Mix (Applied Biosystems, Foster City, Calif., USA), 2.6 μl of Dnase free water and 2.4 μl of each primer set at a final concentration of 300 nM. RT-qPCR was performed using a StepOnePlus Real-Time PCR System (Applied Biosystems) with the following cycling parameters: 95° C. for 20 sec, followed by 40 cycles of 95° C. for 3 sec and 60° C. for 30 sec. This was followed by a dissociation step (95° C. for 15 s, 65° C. for 1 min, 95° C. for 15 sec and finally 60° C. for 15 sec). For each PCR run, a standard curve was generated using five-fold serial dilutions of pooled cDNA. Dissociation curves were examined for each gene to ensure specificity of amplification.

TABLE 4
Reference genes and Target genes analysed by RT-qPCR.

| Gene | | | Amplicon |
|---|---|---|---|
| Symbol | Gene Name | Primer sequence (5′-3′) | Size |
| PPIA | Peptidylprolyl isomerise A | F-GCTCTGAGCACTGGAGAGAAA | 104 |
| | | R-CCATTATGGCGTGTGAAGTC | |
| SDHA | Succinate dehydrogenase complex, | F-TAAACCAAATGCTGGGGAAG | 109 |
| | subunit A, flavoprotein | R-CTGCATCGACTTCTGCATGT | |
| YWHAZ | Tyrosine 3-monooxygenase/tryptophan | F-TGAAGCCATTGCTGAACTTG | 114 |
| | 5-monooxy activation protein, | R-TCTCCTTGGGTATCCGATGT | |
| | zeta polypeptide | | |
| IL1β | Interleukin-1 beta | F-TGATGATGACCTGAGGAGCA | 92 |
| | | R-GTGCGTCACACAGAAACTCG | |
| TNF | Tumour Necrosis Factor | F-GCTCCAGAAGTTGCTTGTGC | 149 |
| | | R-AACCAGAGGGCTGTTGATGG | |
| TLR4 | Toll-Like Receptor 4 | F-AGGCAGCCATAACTTCTCCA | 94 |
| | | R-GCCCTGAAATGTGTCGTCTT | |

### Gene Expression Normalization.

A panel of three reference genes was selected to identify the most stable gene or combination of genes for data normalization of the target genes (Table 4). RT-qPCR was carried out to assess gene transcription levels for all three reference genes at each time point. Analyses of these reference genes were carried out using the geNorm Microsoft Excel add-in. Relative gene expression values were calculated using the standard curve method (Applied Biosystems User Bulletin #2). On the basis of the geNorm analyses, two reference genes, peptidylprolyl isomerase A gene (PPIA) and the tyrosine 3-monooxygenase/tryptophan 5-monooxygenase activation protein, zeta polypeptide gene (YWHAZ), were used to perform the normalization.

### IL-1β and TNF Protein Quantitation.

The levels of the IL-1 beta protein and the TNF protein were measured in the supernatant samples collected from the cell cultures using the bovine IL-1 beta ELISA kit (Thermo Fisher Scientific, Waltham, Mass., USA) and the bovine TNF-Alpha DuoSet ELISA kit (R&D Systems, Inc., Minneapolis, Minn., USA). All assays were carried out as per manufacturer's instructions. Aliquots of the collected culture supernatants were defrosted and 100 μl from each sample was used with each kit to detect the protein levels of IL-1β and TNF present. The IL-1β and TNF protein levels were measured using the Epoch Microplate Spectrophotometer (BioTek Instruments, Inc., Winooski, Vt., USA) plate reader at a wavelength of 450 nm. A set of standards were run on each assay and a value for each sample was derived from the standard curve.

### Statistical Analysis.

A ratio paired t test was used to identify significance differences in gene expression levels between the cell controls and each of the experimental conditions (BHV-1, LPS and BHV-1+LPS) and to identify significance differences in gene expression levels between the different NORS-treated experimental conditions and the corresponding treatment controls. A paired t-test was used to identify significant differences between protein levels of the different experimental conditions.

### Results

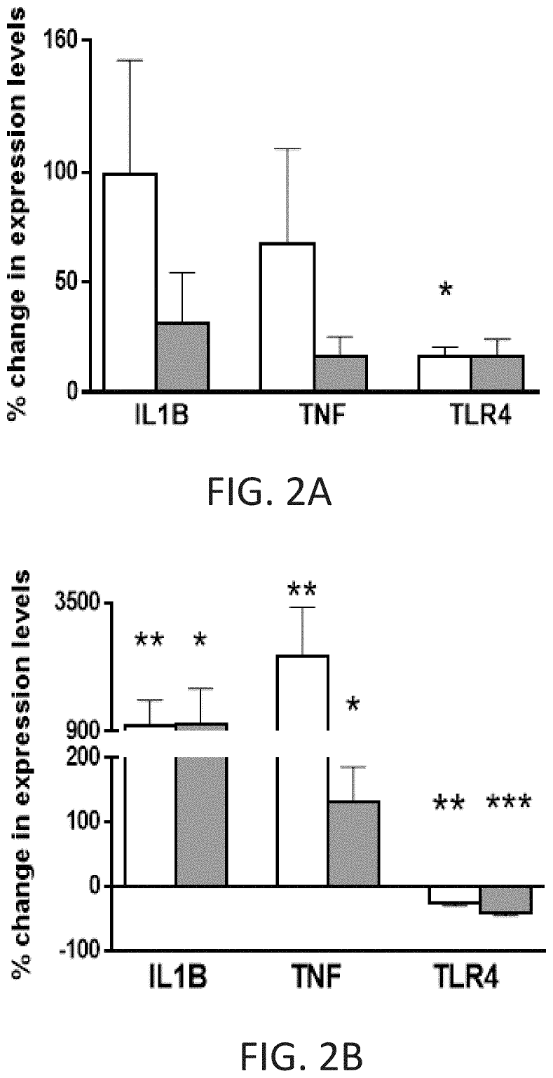

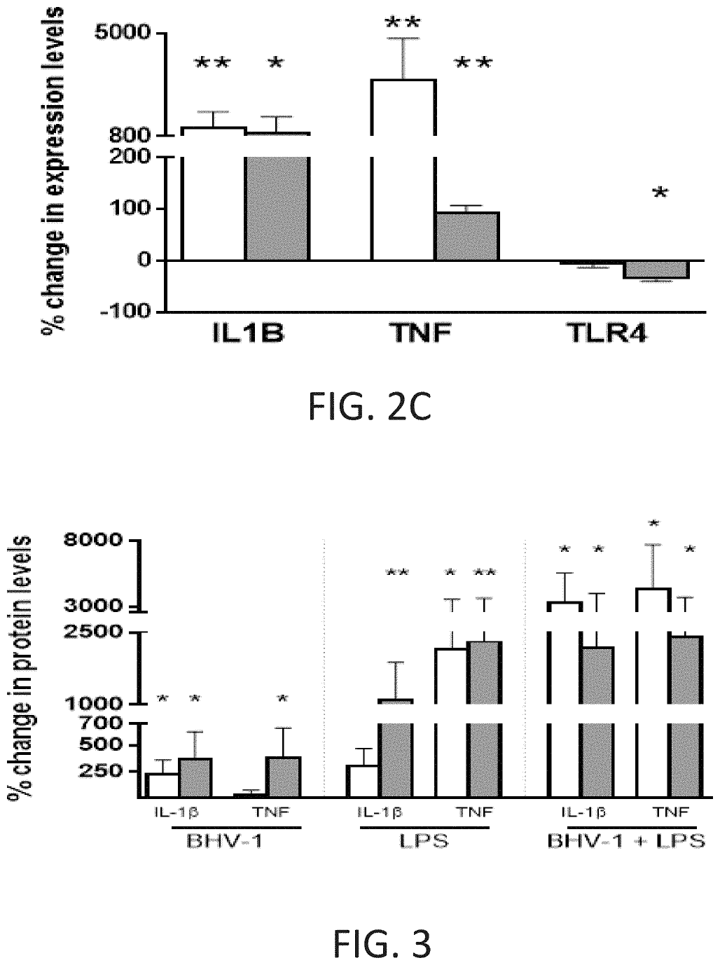

The mRNA and protein immune response of the PBMCs were measured in response to the three different infection conditions. BHV-1 infection alone, of the PBMCs, produced an increased expression of the proinflammatory cytokines IL-1β (99%) and TNF (68%) by T1, when compared against the non-infected PBMC controls (FIG. 2A). This increase had lessened by T2, indicating a reduction in the BHV-1 mediated proinflammatory response. The expression of TLR4 was also increased in response to BHV-1 infection (16%).

The PBMCs cultured alone with LPS displayed significantly greater increases of expression of IL-1β (1026%) and TNF (2440%) (Both p<0.01) at T1, when compared against the non-infected PBMC controls (FIG. 2B). In contrast, the expression of TLR4 was significantly decreased at T1 (−25%) (p<0.01) with a greater decrease noted at T2 (−42%) (p<0.001).

The PBMCs that were both infected with BHV-1 and cultured with LPS (BRDc experimental model) showed even greater increases in IL1β (1174%) (p<0.01) and TNF (3115%) (p<0.01) expression at T1 in comparison with the non-infected controls (FIG. 2C). TLR4 expression was significantly reduced at T2 (−33%) (p<0.05).

The IL-1β and TNF protein levels produced by the cultured bovine PBMCs in response to BHV-1 infection and/or LPS culturing are shown in FIG. 3. The protein levels of IL-1β and TNF were increased significantly (370% and 383% respectively) (Both p<0.05) in response to BHV-1 infection at T2 when compared against the non-infected cell controls. The response to LPS produced greater increases of IL-1β (1102%) and TNF (2301%) (Both p<0.01) when compared with the non-infected cell controls at T2. The increases in protein level in the BRDc experimental model cohort, in which the PBMCs were both infected with BHV-1 and subsequently cultured with the bacterial component LPS, were even greater still at T2: IL-1β (2173%) and TNF (2411%) (Both p<0.05). These protein results demonstrate a similar pattern of regulation to those of the measured mRNA levels of IL-1β and TNF in response to BHV-1 infection and/or LPS.

The host immune response to NORS treatment of bovine PBMCs was investigated using the above in vitro BRDc model consisting of a viral infection and then a latent culturing with bacterial LPS extract. NORS treatment was given following BHV-1 infection but 24 hrs prior to LPS culturing. This intervention point was determined based on the optimal hypothetical time point in the pathogenesis of BRDc.

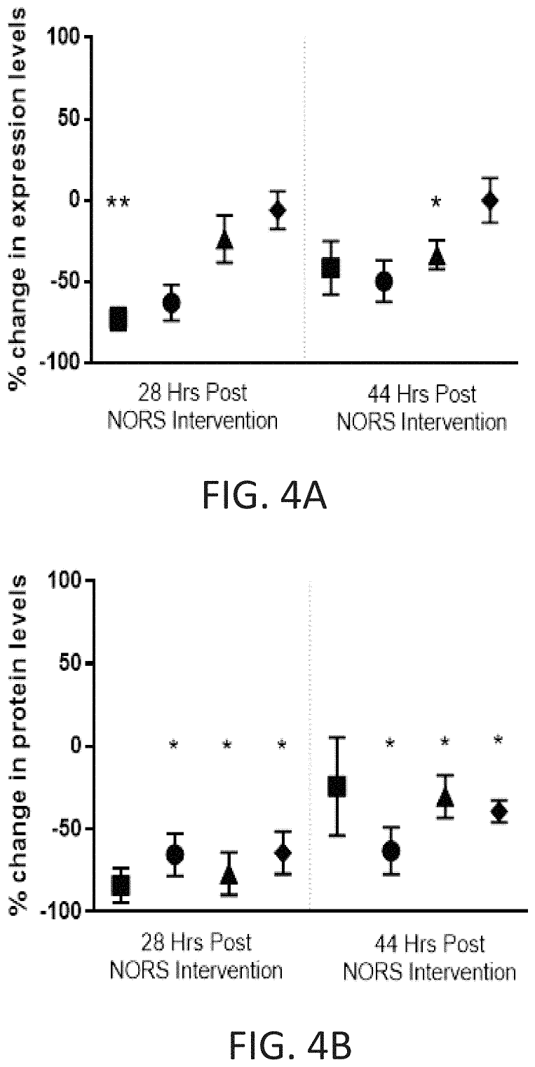

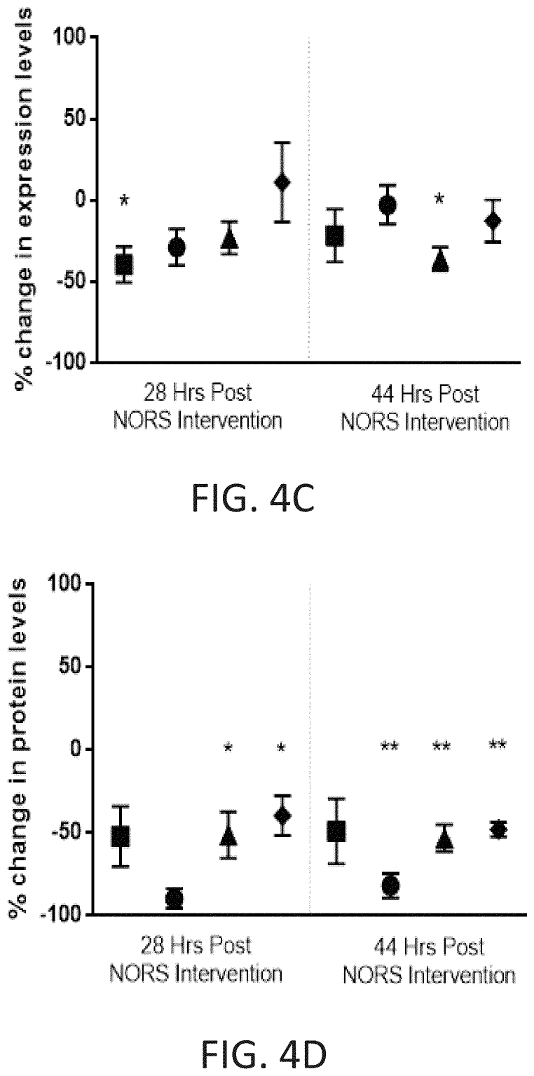

NORS intervention resulted in a 73% reduction of IL1β gene expression in the non-infected cell controls at T1 (p<0.01) (FIG. 4A). In the BHV-1 infected cells, expression was reduced by 63% at T1 with a reduction of 24% in the LPS cultured samples while no reduction was seen in the BRDc experimental samples. At the second time point, T2, the LPS cultured samples showed a greater reduction (−34%) (p<0.05) associated with NORS intervention than at T1.

The protein levels of IL-1β were significantly reduced in the samples treated with NORS in the BHV-1 infected cells at T1 (−65%) (p<0.05) and T2 (−63%) (p<0.05) (FIG. 4B). Moreover, the LPS cultured samples displayed significant decreases at T1 (−77%) and T2 (−30%), (Both p<0.05), while the samples that were both infected with BHV-1 and cultured with LPS, had significant decreases in IL-1β protein at T1 (−65%) and T2 (−39%), (Both p<0.05).

The gene expression pattern of TNF displayed similar levels of reduction in response to NORS as that of IL1β expression. The non-infected cell controls were the only experimental group to be significantly reduced by NORS treatment at T1 (−40%) (p<0.05) while by T2 the LPS cultured samples were the only samples to have significantly reduced TNF expression in response to NORS (−36%) (p<0.05) (FIG. 4C).

The TNF protein results showed significant decreases for the BHV-1 infected cells (−83%) (p<0.01), LPS cultured cells (−54%) (p<0.01) and the BHV-1 infected+LPS cultured cells (−48%) (p<0.01) at T2 in response to NORS (FIG. 4D).

The described gene expression and protein values of IL-1β and TNF demonstrate a pattern of NORS-induced inhibition that is evident in each of the infected experimental conditions.

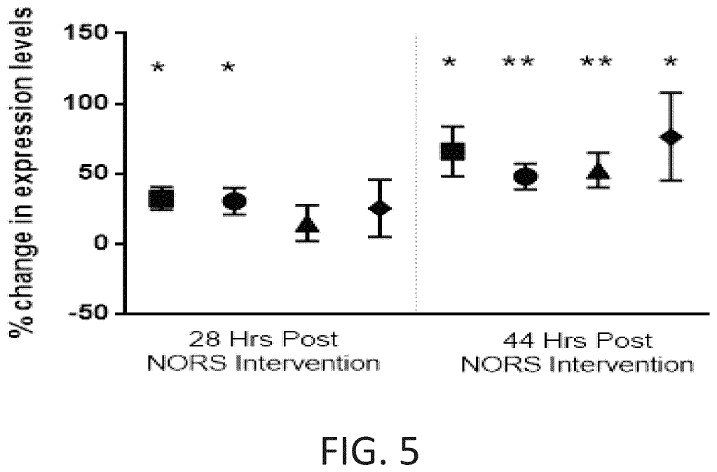

Toll-like receptors are a family of cellular receptors that are fundamental for the recognition of pathogens and subsequent activation of the innate immune response. The Toll-Like Receptor 4 (TLR4) is stimulated by the bacterial component LPS leading to downstream activation of the innate immune response required to defend against bacterial infection. The TLR4 expression levels in the PBMCs treated with NORS were seen to significantly increase in all of the experimental groups by T2 (BHV-1 [48%, p<0.01], LPS [53%, p<0.01], BHV-1+LPS [76%, p<0.05], Non-infected Cell Control [66%, p<0.05]) (FIG. 5). At T1 only the BHV-1 infected (30%) and Non-infected Cell Control (43%) (Both p<0.05) groups displayed significant increases in TLR4 expression. These results suggest a non-specific NORS dependent increase of TLR4 expression under all experimental conditions.

It is noted that one of the purposes of this study was to investigate the innate immune response to an in vitro model of Bovine Respiratory Disease complex (BRDc) and then to examine the role that NO has in modulating that immune response. An in vitro BRDc model was successfully established by using bovine PBMCs that were first infected with BHV-1 and then cultured with LPS extracted from M. haemolytica. This in vitro BRDc model elicited a strong proinflammatory response in the PBMCs while displaying a general suppression of TLR mRNA expression. Treating the virally infected PBMCs with NORS resulted in a significant reduction of the proinflammatory protein response. The enhanced proinflammatory response associated with BHV-1 infection can increase the susceptibility of host cells to M. haemolytica leukotoxin. As such, the reduction in proinflammatory cytokines attributable to the introduction of exogenous NO can provide an explanation of the mechanism to protect the host against bacterial infection and subsequent development of BRDc.

As previously discussed, BRDc is a multifactoral disease that often occurs when active respiratory viral infection increases host susceptibility to M. haemolytica bacterial infection in the lower respiratory tract. While many of the external causes (stressors and pathogens) for BRDc pathogenesis in feedlot cattle are known, the underlying immunological mechanisms of BRDc development are ill defined. The in vitro BRDc model presented in this study allowed a systematic examination of the host cell immune response through the measurement of cellular gene expression and protein production levels. PBMCs were used in this study as they include a highly heterogeneous immune cell population that monitor for and respond to immune-relevant events. While not directly involved in the initial viral infection, which occurs at the respiratory epithelial membrane in the upper respiratory tract, PBMCs will accumulate rapidly at the site of infection and mediate the innate immune response. Thus, examining PBMCs reflect the overall general host immune response as it includes immune effector cells, which are directly involved in the initiation of the adaptive immune response.

BHV-1 viral infection is known to induce an innate proinflammatory response in the host that can last up to 4 days following infection at which stage the cell mediated immune response has been activated and the general immune response becomes more refined. This study shows a similar response in gene expression with both proinflammatory cytokines IL1β and TNF increasing at (>65%) 30 hours post infection then decreasing in expression by 46 hours post infection to levels approaching those of the controls. In contrast, the protein levels of IL-1β and TNF are significantly increased (>350%) 46 hours post BHV-1 infection demonstrating a considerably stronger and prolonged proinflammatory response. Correlation between mRNA and protein levels is generally considered to be poor for a variety of reasons. This disparity in levels can be explained by vast differences in the half lives of individual mRNAs and proteins, while translation ratios can vary possibly producing multiple protein copies from a single mRNA. These factors can, at least in part, explain the differences seen in mRNA and protein levels as a delay between the mRNA and protein signal.

M. haemolytica is the principal bacterium isolated from respiratory diseased cattle in feedlots. M. haemolytica infection produces a strong inflammatory response within hours of bacterial colonization characterized by increased levels of IL-1β and TNF. In this study, LPS extracted from M. haemolytica was used to culture bovine PBMCs, as LPS is a major toxic component of gram-negative bacteria. Culturing with LPS induced an early robust proinflammatory response with IL1β and TNF expression and protein release significantly increased 4 hrs after of the beginning of the LPS culture. This dominant proinflammatory response demonstrates that the isolated M. haemolytica LPS used in this experiment is biologically active and elicits an immune response comparable to that of M. haemolytica infection.

The use of NORS as a preventive agent against the development of BRDc in cattle arriving at the feedlot can be effective under the conditions studied. NO possesses antimicrobial activity against M. haemolytica and BHV-1 in vitro and in vivo. In this study, intervention with NORS also produced a clear pattern of reduced inflammation in all three infection conditions: BHV-1 infection, LPS culturing and a co-culture of BHV-1 infection and LPS culturing of the PBMCs. All three conditions produced significant proinflammatory protein responses at each experimental time point and were significantly reduced in response to the NORS intervention in all cases. These findings suggest that NO (delivered through NORS) can inhibit the development of BRDc through not just a reduction of viral and bacterial load but also with the reduction of inflammation produced during an initial viral infection which has been linked to increased susceptibility to M. haemolytica.

TLR4 is an important initiator of the early innate immune response, as well as the adaptive immune response, that induces expression of inflammatory mediators via signalling pathways following recognition of pathogen associated molecular patterns (PAMPs). These results show that TLR4 expression was significantly increased in BHV-1 infected PBMCs while significantly decreased in the LPS cultured samples when compared against the uninfected control cells. These findings for the LPS cultured samples represents a more unusual result because TLR4 is a well-known recognizer of the bacterial component LPS and is usually increased in its presence. In the model presented here, the LPS mediated suppression of TLR4 expression can, at least in part, be explained as a negative feedback mechanism, to protect against over stimulation due to the high concentrations of LPS used.

The treatment of PBMCs with NORS increased TLR4 mRNA expression levels for all experimental conditions. This could indicate that the response to NORS is non-specific. The response appears to strengthen over the experimental period as the TLR4 mRNA expression levels showed a relative increase between T1 and T2 while gaining greater significance in both the BHV-1 infected and LPS cultured samples. In vulnerable animals with an active viral infection this can provide a protective mechanism against the development of BRDc by providing the host animal with an enhanced ability to detect and respond to bacterial pathogens.

This study has shown how a brief NORS intervention produces an inhibition of the proinflammatory response associated with BRDc, which can provide at least one mechanism for NO mediated host protection against BRDc.

### Example 3

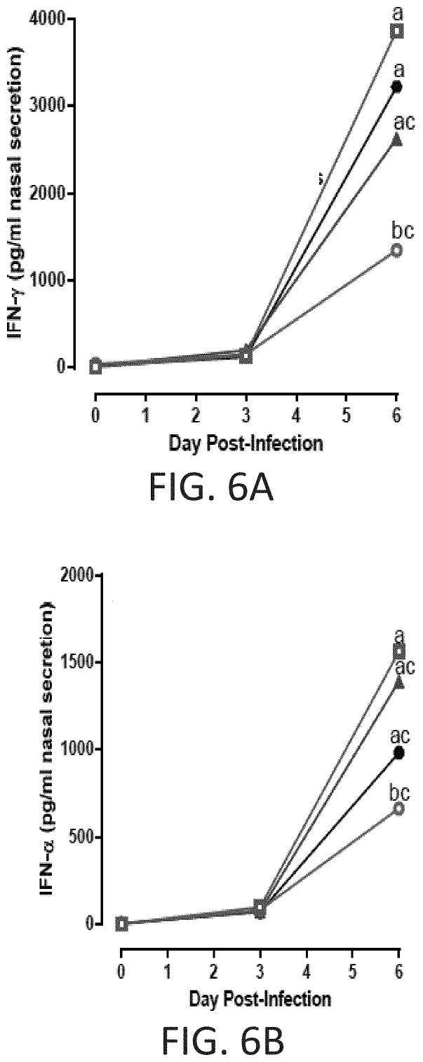

Animals (n=10/group) were treated with either nitric oxide releasing solution (NORS), antibiotic (Draxon), or saline to determine the effect of NORS treatment on the interferon (IFN) response to bovine herpes virus-1 (BHV-1) as compared to antibiotic-treated and control groups. Nasal sections were collected the day prior to BHV-1 infection (Day 0) and on days 3 and 5 post-infection. The levels of IFN-alpha (IFN-α) and IFN-gamma (IFN-γ) were measured using an antibody capture ELISA. FIGS. 6A and 6B illustrate mean IFN-α and IFN-γ levels for the various treatment groups over time.

As illustrated in FIGS. 6A and 6B, IFN-α and IFN-γ secretion increased in all subjects post-infection. Further, antibiotic treatment (black line) had no significant effect on IFN-α and IFN-γ secretion as compared to the control (blue line). However, NORS treatment resulted in significantly (P<0.05) reduced IFN-α and IFN-γ secretion as compared to both control animals and antibiotic-treated animals.

Therefore, because IFN-α can be produced by all nucleated cells in response to viral infection, the presence of IFN-α in nasal secretions following BHV-1 infection can indicate that mucosal epithelial cells in the upper respiratory tract (URT) respond to viral infection by producing IFN-α. However, IFN-γ production can be limited to natural killer (NK) cells and a variety of effector T cells, including γδTcR, CD8, and CD4 T cells. Further, IFN-α can be a potent activator of IFN-γ secretion by NK cells. In the present study, a significant influx of both NK cells and CD8 T cells in the submucosa and mucosa of nasal turbinates were observed on Day 5 post-BHV-1-infection. This can indicate that IFN-α produced by BHV-1 infected mucosal epithelium can activate the recruited NK cells and induce high levels of IFN-γ secretion. This cytokine cascade can also explain 2-fold higher levels of IFN-γ in nasal secretions as compared to IFN-α. Further, this cytokine cascade can explain the observed link between suppression of both IFN-α and IFN-γ levels by NORS treatment.

1. An immunomodulator composition, comprising an amount of a liquid nitric oxide releasing solution (NORS) sufficient to elicit an immune response in a subject that is adequate to treat an adverse health condition in the subject.

2. The immunomodulator composition of claim 1, further comprising a biological agent.

3. The immunomodulator composition of claim 2, wherein the biological agent is selected from the group consisting of an immune enhancer protein, an immunogen, a vaccine, an antimicrobial, and combinations thereof.

4. The immunomodulator composition of claim 1, wherein the adverse health condition includes at least one of a viral infection and a bacterial infection.

5. The immunomodulator composition of claim 1, wherein the amount of NORS is sufficient to increase expression of a toll-like receptor in the subject at a target location within a predetermined period as compared to an untreated subject.

6. The immunomodulator composition of claim 5, wherein expression of the toll-like receptor is increased by at least 30%.

7. The immunomodulator composition of claim 5, wherein the toll-like receptor comprises toll-like receptor 3, toll-like receptor 4, or a combination thereof.

8. The immunomodulator composition of claim 5, wherein the predetermined period is within 4 hours.

9. The immunomodulator composition of claim 5, wherein the predetermined period is within 20 hours.

10. The immunomodulator composition of claim 1, wherein the amount of NORS is sufficient to reduce an amount of a proinflammatory protein present in the subject at a target location within a predetermined period as compared to an untreated subject.

11. The immunomodulator composition of claim 10, wherein the amount of proinflammatory protein is reduced by at least 30%.

12. The immunomodulator composition of claim 10, wherein the proinflammatory protein is selected from the group consisting of interleukin 1 beta, interleukin 8, interleukin 10, tumor necrosis factor alpha, and combinations thereof.

13. The immunomodulator composition of claim 10, wherein the predetermined period is within 4 hours.

14. The immunomodulator composition of claim 10, wherein the predetermined period is within 20 hours.

15. The immunomodulatory composition of claim 1, wherein the amount of NORS is sufficient to reduce an amount of an acute-phase protein present in the subject within a predetermined period as compared to an untreated subject.

16. The immunomodulatory composition of claim 15, wherein the amount of acute-phase protein is reduced by at least 30%.

17. The immunomodulatory composition of claim 15, wherein the acute-phase protein comprises haptoglobin.

18. The immunomodulatory composition of claim 15, wherein the predetermined period is within 10 days.

19. A method of eliciting an immune response in a subject, comprising administering to the subject a therapeutically effective amount of a NORS.

20-33. (canceled)

34. A method of eliciting an immune response, or improving an acquired immune response in a subject, comprising administering to the subject, a therapeutically effective amount of an immunomodulatory composition as recited in claim 1.

35. (canceled)
